(19) United States
(12) Patent Application Publication
(10) Pub. No.: US 2016/0045574 A1
(43) Pub. Date: Feb. 18, 2016

(54) ANTIVIRAL PHARMACEUTICAL FOR TOPICAL ADMINISTRATION

(71) Applicant: TAMIR BIOTECHNOLOGY, INC., San Diego, CA (US)

(72) Inventors: Jamie Sulley, Old Saybrook, CT (US); Luis Squiquera, Buenos Aires (AR)

(73) Assignee: TAMIR BIOTECHNOLOGY, INC., San Diego, CA (US)

(21) Appl. No.: 14/462,520

(22) Filed: Aug. 18, 2014

(51) Int. Cl. A61K 38/46

(52) U.S. Cl. CPC A61K 38/465; C12Y 301/04

(57) ABSTRACT

An enzymatically-active ribonuclease is combined with a vehicle that does not unacceptably interfere with such enzymatic activity and applied externally. Advantageously, the ribonuclease is ranpirnase. The vehicle can be a liquid, a gel, an ointment, or a serum, and can also be an approved sexual lubricant.

### BACKGROUND OF THE INVENTION

The invention relates to antiviral pharmaceuticals, and more particularly relates to antiviral pharmaceuticals for topical administration. In its most immediate sense, the invention relates to pharmaceuticals for treating patients with anogenital warts.

Ranpirnase is a protein with ribonuclease activity, it has a molecular weight of approximately 12,000 Daltons, and it has an amino acid sequence disclosed and claimed in U.S. Pat. No. 5,559,212. It can be isolated from embryos and eggs of the Rana pipiens frog or produced as a recombinant protein (see e.g. U.S. Pat. No. 6,175,003 B1). Commonly-owned patent number U.S. Pat. No. 8,663,964 B2 teaches that ranpirnase and another enzymatically-active ribonuclease are active against human papillomavirus (hereinafter, “HPV”) and that HPV can be treated by using either of these two ribonucleases on an HPV-infected region of a patient. Anogenital warts are caused by various human papillomaviruses, and no satisfactory treatment exists for this sexually-transmitted disease since all available treatment modalities target the lesions and lack viricidal activity against HPV.

It has been proposed to treat anogenital warts intralesionally, i.e. by injecting an active pharmaceutical ingredient (“API”) into the wart to be treated. In many situations—where the warts are small or too numerous, or in locations where an injection would be too painful—this would be unsatisfactory.

It has also been proposed to treat anogenital warts topically. Because the HPV-infected cells are located beneath the surface of the patient's skin and would not be directly contacted by the API, this proposal assumed that it would be necessary to administer the API using a special vehicle that would penetrate through the intervening layers of the patient's skin to thereby deliver the API to a location where its anti-HPV activity would be useful

A compassionate use study presently being carried out on male volunteers with anogenital warts has produced an unexpected and surprising result that demonstrates this assumption to be incorrect. In this two-arm study, ranpirnase was combined with two different vehicles and the combination was applied topically to HPV lesions.

From study results to date, it now appears that topical ranpirnase therapy for HPV does not—as was expected—require a vehicle having penetrating characteristics. Rather, it appears that the vehicle need only not unacceptably interfere with the enzymatic activity of ranpirnase. And, because there other enzymatically—active ribonucleases that behave similarly to ranpirnase it is reasonable to expect that any such ribonuclease will, when combined with a suitable vehicle, have an activity similar to that of ranpirnase.

This compassionate use study is ongoing and it is presently possible to draw only an interim conclusion from it. But, this interim conclusion is that the invention appears to be effective in the treatment and suppression of anogenital warts.

Significantly, it appears that the vehicle need not be entirely free of anti-enzymatic activity. In some instances, it is believed possible to overcome anti-enzymatic qualities of the carrier by increasing the concentration of the enzymatically-active ribonuclease.

In accordance with the invention, a pharmaceutical comprises a therapeutically effective quantity of an enzymatically-active ribonuclease and a vehicle that does not unacceptably interfere with such enzymatic activity.

Preferred embodiments of the invention use ranpirnase as the enzymatically-active ribonuclease. While an oil in water -based vehicle containing other components such as collagen can be used, preferred embodiments use an aqueous vehicle. Preferred aqueous vehicles are gels, serums, lotions, or approved sexual lubricants (which may themselves be gels or lotions). This is because gels, lotions, serums, and sexual lubricants are viscous or can be made viscous so that the invention will remain where it has been applied and will not run off. Advantageously, and in accordance with preferred embodiments of the invention, the pharmaceutical has between 1 and 3 mg of ranpirnase per ml of vehicle.

### BRIEF DESCRIPTION OF THE DRAWING

FIG. 1 shows the clinical responses of seventeen subjects in a presently ongoing compassionate use study.

### DETAILED DESCRIPTION OF PREFERRED EMBODIMENTS

A presently-ongoing compassionate use study is being conducted. The study is being conducted on male volunteers who have been diagnosed with anogenital warts in various locations (scrotum, penis shaft, penis dorsum, inguinal, perianal). This study has two arms, each testing the effect of a single one of the two embodiments of the invention disclosed herein. In one arm, the tested embodiment is ranpirnase combined with a vehicle supplied by JRX Biotechnology, Inc. This first embodiment is a very low-viscosity liquid; it is applied topically twice each day. The vehicle is a polysaccharide megasphere formulation; on information and belief it is covered by U.S. Pat. Nos. 6,759,056, 6,946,144, 7,201,919, 7,220,427, 7,300,666, and 7,316,820. In the other arm (which serves as a control arm), the tested—and presently preferred—embodiment is ranpirnase combined with K-Y® Brand Jelly; this second (and preferred) embodiment is a viscous gel that is being applied topically three times each day. In each arm, the concentration of ranpirnase is 1 mg ranpirnase to 1 ml of vehicle.

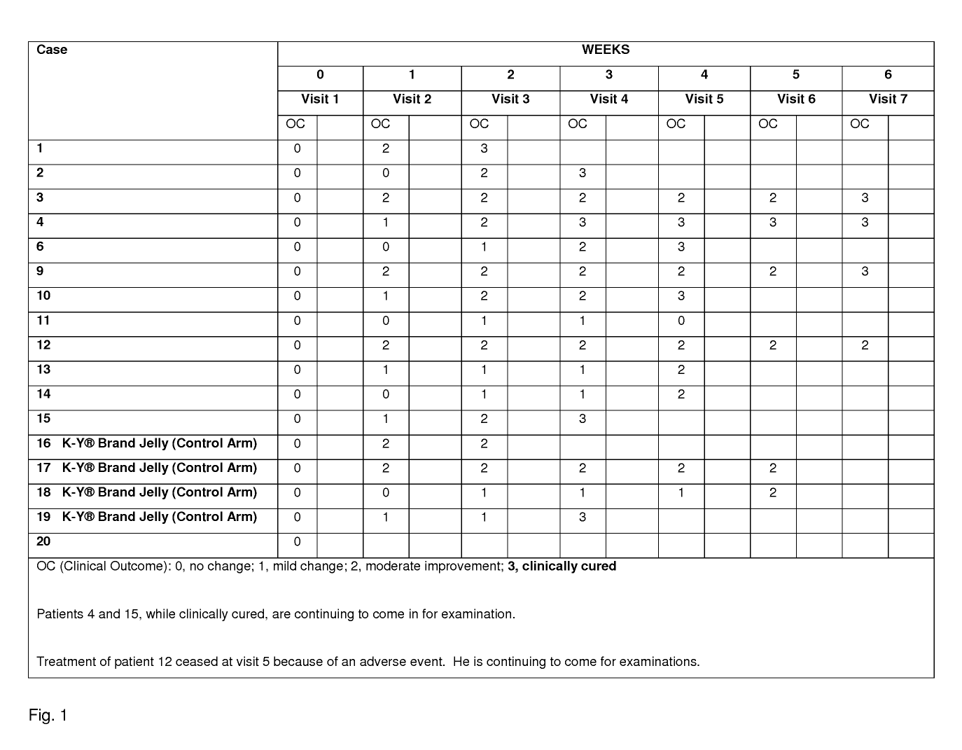

As can be seen in FIG. 1, of the thirteen evaluated subjects who received the first embodiment, seven achieved complete remission by the seventh visit. Although the long-term effect of this treatment is not yet known, this short-term effect is far better than has been reported for other pharmaceuticals (Podofilox solution and gel, Aldara Cream, Veregen ointment).

In addition to being more viscous than the first embodiment of the invention, the second (and preferred) embodiment of the invention may be slightly more active than the first embodiment. Results to date show that in 50% of the four subjects treated with the preferred embodiment, there was more than a 50% improvement by the second visit. And, one of the subjects achieved complete remission by the fourth visit. These results are an improvement over the clinical response observed in the first embodiment. It is believed that the activity of the preferred embodiment can be further increased by increasing the concentration of ranpirnase from 1 mg/ml to 3 mg/ml.

It will be understood that although the invention has been developed for use in treatment of anogenital warts, its use is not restricted to this application. The invention may have other antiviral applications. Although the invention is presently applied to external anogenital warts, it may also be useful when applied vaginally, extra-vaginally, intra-vaginally, anally, peri-anally, and intra-anally. Although the preferred embodiment uses ranpirnase as the enzymatically-active ribonuclease, other ribonucleases having similar enzymatic activities exist and may be used instead. Furthermore, while treatment of anogenital warts may be easier using a viscous vehicle such as a gel because it is easier to apply a gel to an anogenital wart and a gel is less likely to run off and is therefore more likely to remain where it has been applied, there may be other applications in which a liquid vehicle, or an ointment vehicle will be preferable. The vehicle may alternatively be a serum, a lotion, or an approved sexual lubricant, i.e. a lubricant that meets applicable governmental requirements and is intended for use on human genitalia.

Although at least one preferred embodiment of the invention has been described above, this description is not limiting and is only exemplary. The scope of the invention is defined only by the claims, which follow:

1. A pharmaceutical comprising a therapeutically effective quantity of an enzymatically-active ribonuclease and a vehicle that does not unacceptably interfere with such enzymatic activity.

2. The pharmaceutical of claim 1, wherein the enzymatically-active ribonuclease is ranpirnase.

3. The pharmaceutical of claim 1, wherein the vehicle is an aqueous vehicle.

4. The pharmaceutical of claim 3, wherein the vehicle is a gel, a serum, or a lotion.

5. The pharmaceutical of claim 3, wherein the vehicle is an approved sexual lubricant.

6. The pharmaceutical of claim 1, wherein the vehicle is compatible with latex condoms.

7. The pharmaceutical of claim 2, wherein the pharmaceutical contains between 1 and 3 mg of ranpirnase per ml of vehicle.
